(19) United States
(12) Patent Application Publication
(10) Pub. No.: US 2026/0108320 A1
(43) Pub. Date: Apr. 23, 2026

(54) DEVICES, SYSTEMS, AND METHODS OF POSITIONING BONE SEGMENTS

(71) Applicant: Rowan University, Glassboro, NJ (US)

(72) Inventors: Mohammad Abedin-Nasab, Cherry Hill, NJ (US); Marzieh Saeedi-Hosseiny, Cherry Hill, NJ (US)

(73) Assignee: Rowan University, Glassboro, NJ (US)

(21) Appl. No.: 19/106,431

(22) PCT Filed: Jul. 11, 2024
(86) PCT No.: PCT/US2024/037613

§ 371 Date: Feb. 25, 2025

Related U.S. Application Data
(60) Provisional application No. 63/512,941, filed on Jul. 11, 2023.

(51) Int. Cl. A61B 90/00; A61B 6/40; A61B 6/50; A61B 34/20

(52) U.S. Cl. CPC A61B 90/39; A61B 6/4057; A61B 6/505; A61B 34/20; A61B 2034/2055; A61B 2090/3916; A61B 2090/3937; A61B 2090/3966; A61B 2090/3983

(57) ABSTRACT

A device for orienting bone fragments is provided herein. The device includes a support element, at least three positional markers disposed on the support element, and at least three optical tracking markers disposed on the support element. Also provided herein are a system including the device and a method of monitoring bone fragment positioning in real-time using the device.

### CROSS-REFERENCE TO RELATED APPLICATIONS

The present application claims priority under 35 U.S. C. § 119(e) to U.S. Provisional Ser. No. 63/512,941 , filed Jul. 11, 2023, which application is incorporated herein by reference in its entirety.

### BACKGROUND

Long-bone fractures are a common occurrence in traumatic injury incidents where a rigorous process is required to fix the fracture. Manual techniques are currently used for fraction reduction surgery. There is no commercially available imaging system available for long-bone fracture alignment which can be used to track the 3D position of bone fragments during a realignment procedure.

Accordingly, there is a need in the art for articles and methods that improve on existing articles and methods of fracture alignment. The present invention addresses this need.

### SUMMARY

In one aspect, provided herein is a device for orienting bone fragments including a support element; at least three positional markers disposed on the support element; and at least three optical tracking markers disposed on the support element.

In some embodiments, the at least three positional markers comprise a computed tomography (CT) scan-detectable material. In some embodiments, the material of the positional markers is distinguishable from bone in the CT scan. In some embodiments, each of the positional markers is a radiopaque marker. In some embodiments, each of the positional markers is spherical. In some embodiments, the device further includes a containment structure for each of the positional markers. In some embodiments, the containment structure is configured to optically conceal each of the positional markers. In some embodiments, the at least three optical tracking markers comprise infrared (IR)-detectable materials.

In some embodiments, the at least three positional markers are fixed relative to the at least three optical tracking markers on the support element. In some embodiments, the optical tracking markers are radially distal to the positional markers with respect to a substantially central point on the support element. In some embodiments, at least one of the positional markers is fixed in a different plane from at least one of the remaining positional markers; and at least one of the optical tracking markers is fixed in a different plane from at least one of the remaining optical tracking markers. In some embodiments, at least one of the positional markers is fixed asymmetrically in at least one dimension with respect to the remaining positional markers; at least one of the optical tracking markers is fixed asymmetrically in at least one dimension with respect to the remaining optical tracking markers; or a combination thereof.

In some embodiments, the support element comprises a fixed frame. In some embodiments, the fixed frame includes an orthopedic screw attached thereto. In some embodiments, the support element comprises a surgical robot.

In another aspect, provided herein is a system including at least one device according to any of the embodiments disclosed herein; and a camera system configured and adapted to detect one or more optical tracking markers. In some embodiments, the at least one device includes a first device configured to be installed to a proximal bone fragment and a second device configured to be installed to a distal bone fragment. In some embodiments, the system further includes a computer system configured and adapted to communicate with the camera system, wherein the computer system is further configured and adapted to implement an optical tracking algorithm.

In a further aspect, provided herein is a method of monitoring bone fragment positioning in real-time, the method including the steps of (a) attaching a first device to a proximal bone fragment, the first device comprising a device according to any of the embodiments disclosed herein; (b) attaching a second device to a distal bone fragment, the second device comprising a device according to any of the embodiments disclosed herein; (c) obtaining imaging of the bone fragments with the attached first and second devices; (d) determining a position of the first device with respect to the proximal bone fragment based upon the imaging; (e) determining a position of the second device with respect to the distal bone fragment based upon the imaging; (f) monitoring the optical tracking markers of the first device and the second device using infrared (IR) cameras during at least a portion of a surgery; and (g) determining a position of the distal bone fragment relative to the proximal bone fragment in real-time based upon the monitoring of the optical tracking markers.

In some embodiments, the imaging in step (c) includes a computed tomography (CT) scan. In some embodiments, the surgery comprises manual or robotic surgery. In some embodiments, the portion of the surgery in step (f) comprises a repositioning operation, the repositioning operation including moving one or more of the distal bone fragment and the proximal bone fragment relative to each other. In some embodiments, the method further includes (h) confirming a position of the proximal bone fragment with respect to the distal bone fragment. In some embodiments, the confirming step includes collecting an X-ray image.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

The invention is best understood from the following detailed description when read in connection with the accompanying drawings. It is emphasized that, according to common practice, the various features of the drawing are not to scale. On the contrary, the dimensions of the various features are arbitrarily deformed or reduced for clarity. Included in the drawings are the following figures.

FIGS. 1A-1B illustrate a device for orienting bone fragments, in accordance with certain exemplary embodiments of the present disclosure.

FIGS. 2A-2B illustrate a system for orienting bone fragments, in accordance with certain exemplary embodiments of the present disclosure.

FIG. 3 illustrates a graphical user interface (GUI) illustrating 3D representations of bone fragments, in accordance with certain exemplary embodiments of the present disclosure.

FIG. 4 illustrates a system for orienting bone fragments, in accordance with certain exemplary embodiments of the present disclosure.

### DETAILED DESCRIPTION

### Definitions

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,”“including,”and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION

Provided herein are devices for orienting bone fragments. In some embodiments, the device includes a support element, at least three positional markers disposed on the support element, and at least three optical tracking markers disposed on the support element. In some embodiments, each device is arranged and disposed to be attached to a bone fragment. In such embodiments, attaching the device to a bone fragment fixes the positional markers and the optical tracking markers with respect to that bone fragment.

The positional markers include any shape and/or material suitable for detection with x-ray imaging, computed tomography (CT) imaging, or any other imaging capable of detecting the position and orientation of the bone fragments. For example, in some embodiments, the positional marker is radiopaque. Additionally or alternatively, in some embodiments, the positional marker is spherical. In some embodiments, the positional marker is distinguishable from bone in the relevant imaging. In some embodiments, the positional marker includes a material that is not optically detectable. Alternatively, in some embodiments, the device includes a containment structure (e.g., cap, cover, shell, etc.) for each of the positional markers. In such embodiments, the containment structure is configured to optically conceal each of the positional markers.

The optical tracking marker includes any shape and/or material suitable for optical detection. For example, in some embodiments, the optical tracking marker includes an infrared (IR)-detectable material. In some embodiments, the optical tracking markers are larger than the positional markers. Additionally or alternatively, the optical tracking markers may include the same or different shapes as the positional markers.

The positional markers and the optical tracking markers are fixed and/or can be adjustably fixed to the support element. In some embodiments, the positional markers and/or the optical tracking markers are fixed relative to each other and/or an origin point (e.g., central or substantially central point) on the support element. In some embodiments, the optical tracking markers are radially distal to the positional markers with respect to a substantially central point on the support element. In some embodiments, at least one of the positional markers and/or optical tracking markers is fixed in a different plane from at least one of the other positional markers and/or optical tracking markers. In some embodiments, at least one of the positional markers and/or optical tracking markers is fixed asymmetrically in at least one dimension/direction with respect to at least one of the other positional markers and/or optical tracking markers.

Although described herein primarily with respect to three or four of each type of marker, as will be appreciated by those skilled in the art, the disclosure is not so limited and may include any suitable number of each marker, including, but not limited to, at least 5, at least 6, at least 7, or more of each marker. As will also be appreciated by those skilled in the art, while the device is primarily described with respect to the same number of positional and optical tracking markers, the disclosure is not so limited and explicitly includes embodiments with different numbers of positional and optical tracking markers.

The support element includes any article arranged and disposed to fix the positional and optical tracking markers relative to a bone fragment. For example, in some embodiments, the support element includes a fixed frame having one or more segments arranged and disposed to fix a position of the positional and/or optical tracking markers relative thereto. Alternatively, in some embodiments, the support element includes a surgical robot, such as, but not limited to, the surgical robot according to any of the embodiments disclosed in U.S. U.S. Pat. No. 10,603,122, which is incorporated herein by reference in its entirety. As will be appreciated by those skilled in the art, the support element may include any suitable shape for providing the desired fixation of the markers. In some embodiments, the fixation of markers in asymmetrically and/or in different planes increases the viewing angle as compared to markers positioned symmetrically and/or in a single plane.

The support element may be attached/fixed relative to a corresponding bone fragment in any suitable manner. In some embodiments, a rod (e.g., a threaded rod, a screw, an orthopedic screw, etc.) is used to attach the device to a bone fragment. In such embodiments, the rod can be unicortical (e.g., unicortical locking screws) or bicortical (e.g., bicortical locking screws). For example, in some embodiments, the support element (e.g., fixed frame) includes an orthopedic screw attached thereto and/or is arranged and disposed to couple with an orthopedic screw. Additionally or alternatively, in some embodiments, one or more markers can be attached directly to the skin and/or using one or more traction rods. In some embodiments, the configuration of the markers creates an asymmetric reference element with at least two orthogonal hands, while the center of the geometry of each element is located on the conjunction of four hands.

Also provided herein are systems for orienting bone fragments. In some embodiments, the system includes at least one device according to any of the embodiments disclosed herein, and a camera system configured and adapted to detect one or more optical tracking markers. In some embodiments, the system further includes a computer system configured and adapted to communicate with the camera system. In some embodiments, the computer system is configured and adapted to implement an optical tracking algorithm.

The system according to one or more of the embodiments disclosed herein may be used to track the three-dimensional (3D) position of one or more bone fragments in real-time. For example, in some embodiments, the system is arranged and disposed to receive imaging from one or more scans (e.g., x-rays, CT scans, etc.) and determine the position of the at least one device with respect to a corresponding bone fragment based upon the orientation of the positional markers. The optical tracking markers of the rigid bodies can then be registered to the positional markers that are visible in the CT scan (e.g., in consideration of the offset between an optical tracking marker and a respective positional marker). After the position of the device is determined with respect to the corresponding bone fragment and the optical tracking markers have been registered to the positional markers, the camera system can be used to monitor the optical tracking markers. Since the optical tracking markers are fixed relative to the positional markers, and the positional markers are fixed relative to the bone fragment, the positioning and orientation of the bone fragment can be viewed/determined in real-time based upon the position and orientation of the optical tracking markers.

In some embodiments, the system only uses a single device according to any of the embodiments disclosed herein to determine the relative position of a corresponding bone fragment. For example, in some embodiments, the single device is attached to a first bone fragment (e.g., a distal fragment), while the position/orientation of a second bone fragment (e.g., a proximal fragment) is determined through registration of landmarks (e.g., distinct or distinguishable features).

Alternatively, in some embodiments, one or more separate devices may be attached to individual bone fragments (e.g., a first device to a first bone segment, a second device to a second bone segment, etc.). For example, in some embodiments, the system includes a first device configured to be installed to a proximal bone fragment and a second device configured to be installed to a distal bone fragment. Each device may be individually and independently attached to the corresponding bone fragment in the same or a different manner as compared to any of the other devices. In some embodiments, the system include at least one additional optical tracking camera for multiple devices. For example, in some embodiments, the system includes two devices, a camera system including an optical tracking dual-camera, and optical motion capture software.

The optical tracking algorithm according to any of the embodiments disclosed herein can automatically determine the positioning/orientation of the bone fragment based upon the position of the optical tracking markers as monitored by the camera system. In some embodiments, the system includes a GUI configured to display the real-time 3D position of the bone fragments to a surgeon while the fragments are being manipulated and/or fixed. Additionally or alternatively, the software may be coupled with a robotic system to align long-bone fractures. For example, an auto-alignment algorithm may find the optimum path to guide the robot to manipulate bone fragments from the initial fracture position to the final desired position obtained from the landmarks of the intact contralateral bone.

Further provided herein are methods of positioning bone fragments. In some embodiments, the method includes attaching at least one device according to any of the embodiments disclosed herein to at least one corresponding bone fragment, obtaining imaging of the bone fragments and the at least one attached device, determining a position of the at least one device with respect to the corresponding bone fragment, monitoring the optical tracking markers of the at least one device with an optical camera during at least a portion of a surgery; and determining a relative position of the bone fragments in real-time based upon the monitoring of the optical tracking markers. In some embodiments, the imaging includes an x-ray, a computed tomography (CT) scan, or the like. The surgery may be manual or robotic surgery, and includes any procedure for reducing, positioning, fixing, and/or otherwise treating a fracture. For example, in some embodiments, the surgery includes a repositioning operation involving moving one or more of the distal bone fragment and the proximal bone fragment relative to each other. In some embodiments, the method further includes confirming a position of the bone fragments following manipulation/repositioning. In some embodiments, the confirming step includes collecting an X-ray image.

In some embodiments, the method includes attaching a single device to a first bone fragment, registering landmarks of a second bone fragment, and monitoring the position of the bone fragments in real-time based upon the optical tracking markers of the device attached to the first bone fragment and the registered landmarks of the second bone fragment.

Alternatively, in some embodiments, at least one device is separately and individually attached to two or more bone fragments. For example, in some embodiments, the method includes (a) attaching a first device according to any of the embodiments disclosed herein to a proximal bone fragment; (b) attaching a second device according to any of the embodiments disclosed herein to a distal bone fragment; (c) obtaining imaging of the bone fragments with the attached first and second devices; (d) determining a position of the first device with respect to the proximal bone fragment based upon the imaging; (e) determining a position of the second device with respect to the distal bone fragment based upon the imaging; (f) monitoring the optical tracking markers of the first device and the second device using infrared (IR) cameras during at least a portion of a surgery; and (g) determining a position of the distal bone fragment relative to the proximal bone fragment in real-time based upon the monitoring of the optical tracking markers. As will be appreciated by those skilled in the art, when more than two fragments are present, one or more devices may be attached to each fragment, or a combination of devices and landmarks may be used to determine the position/orientation of all fragments.

To estimate the results of alignment and how much the deviation from the final desired alignment is, the real-time relative position of fractured long-bone segments can be compared to the final anatomical position, which can be captured from unbroken bone data (e.g., prior imaging and/or the opposite bone). In some embodiments, deviations in 6 degrees of freedom can be shown in a designed GUI (e.g., using six gauges). When the deviation of position and rotation from the alignment is less than the defined limits, the respective gauge can turn green. Such a color change can notify the surgeon that the accurate alignment is complete.

In some embodiments, the method includes obtaining a CT scan of both the fractured long-bone with one or more devices attached thereto and of the contralateral unfractured long-bone. In some embodiments, the GUI displays the 3D scan of the healthy and fractured long-bones. In some embodiments, specific landmarks on the distal and proximal long-bone fragments may be input manually (e.g., using a touch screen on the GUI) intraoperatively. Using the input landmarks on the “healthy bone” (e.g., an opposite/mirrored bone which is not fractured), a developed algorithm can calculate the lengths and rotations of the desired anatomical alignment for the fractured bone. Alternatively, in some embodiments, alignment of the bone fragments can be calculated without input landmarks from a “healthy bone.” For example, alignment can be calculated based on (at least in part) matching surfaces (e.g., the fracture surface) of the bone fragments.

In contrast to existing manual techniques for fraction reduction surgery, which involve rigorous processes and extensive repeated x-ray exposure, the devices, systems, and methods described herein facilitate and/or provide imaging software can be used to help accurately align bone fragments in real-time or near-real-time. Using such software in connection with devices and systems described herein, surgeons can track the 3D position and rotation of the fractured bone during surgery.

The following examples further illustrate aspects of the present invention. However, they are in no way a limitation of the teachings or disclosure of the present invention as set forth herein.

### EXAMPLES

Example 1

### Device for Orienting Bone Fragments

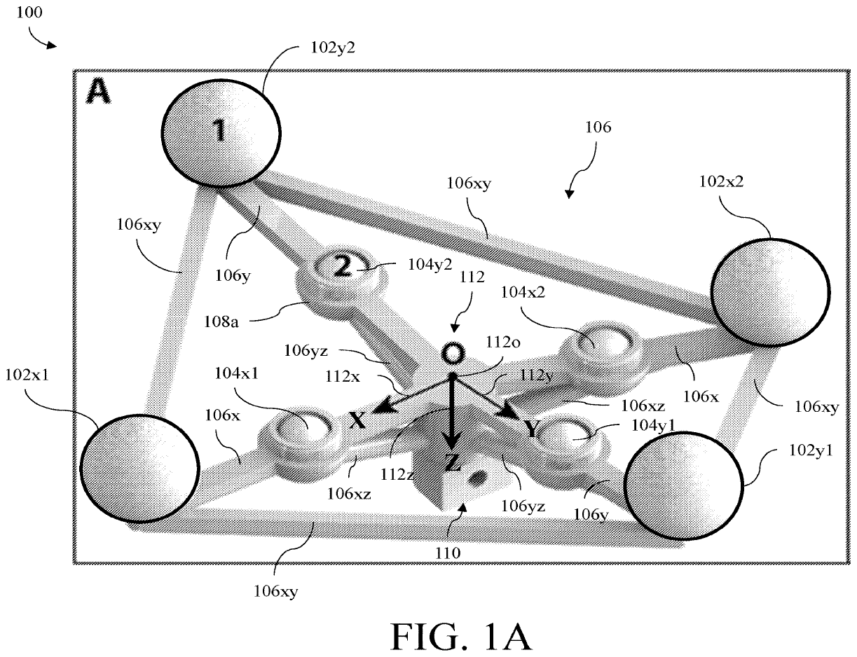

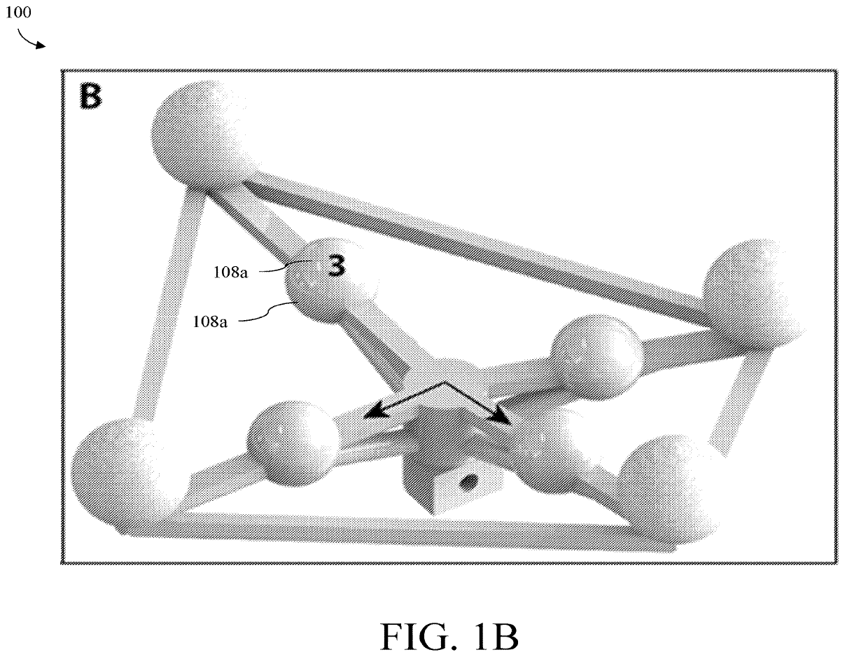

Referring now to the drawings, in FIGS. 1A-1B, a device 100 for orienting bone fragments is illustrated. Device 100 includes a frame 106 (e.g., a fixed frame, a rigid frame). Frame 106 provides sufficient fixity and/or rigidity such that a marker (e.g., an optical tracking marker, a radiopaque marker, etc.) disposed thereon retains an effectively constant positional relationship to other markers disposed thereon. Accordingly, a “fixed frame” as used herein refers to a frame which includes frame components which are fixed relative to each other as the frame translates in space (e.g., during an alignment procedure/operation). Frame 106 is illustrated defining a local coordinate system 112. Local coordinate system 112 defines an origin 1120, a first axis 112x (e.g., an X-axis), a second axis 112y (e.g., a Y-axis), and a third axis 112z (e.g., a Z-axis). First axis 112x, second axis 112y, and third axis 112z are illustrated being orthogonal to each other and positioned at origin 1120.

Device 100 also includes a positional marker 104y1 (e.g., a radiopaque marker, a spherical radiopaque marker, a non-optically detected marker, etc.) disposed at a distance from origin 1120. Positional marker 104y1 is illustrated disposed at a distance substantially along the Y-axis. However, it should be noted that positional marker 104y1 can include a positional “Z component” and/or an “X component” (i.e., as defined by local coordinate system 112).

Device 100 is illustrated including a plurality of positional markers (i.e., positional markers 104y1, 104y2, 104x1, and 104x2). Although each of the positional markers 104y1, 104y2, 104x1, and 104x2 appear to be symmetrically distributed (e.g., where 104y1 and 104y2 appear to be mirrored across an XY-plane, the plane defined by the X-axis and Z-axis), the invention is not so limited. In certain embodiments, positional markers 104y1, 104y2, 104y1, and 104y2 define an asymmetric configuration (e.g., with respect to one or more axes, with respect to one or more planes defined by the axes, etc.).

Device 100 also includes an optical tracking marker 102y1 disposed at a distance from origin 1120. Optical tracking marker 102y1 is illustrated disposed at a distance substantially along the Y-axis. However, it should be noted that optical tracking marker 102y1 can include a positional “Z component” and/or an “X component” (i.e., as defined by local coordinate system 112).

Device 100 is illustrated including a plurality of optical tracking markers (i.e., optical tracking markers 102y1, 102y2, 102x1, and 102x2). Although each of the optical tracking markers 102y1, 102y2, 102x1, and 102x2 appear to be symmetrically distributed (e.g., where 102y1 and 102y2 appear to be mirrored across an XY-plane, the plane defined by the X-axis and Z-axis), the invention is not so limited. In certain embodiments, optical tracking markers 102y1, 102y2, 102x1, and 102x2 define an asymmetric configuration (e.g., with respect to one or more axes, with respect to one or more planes defined by the axes, etc.).

### Example 2

### System for Orienting Bone Fragments

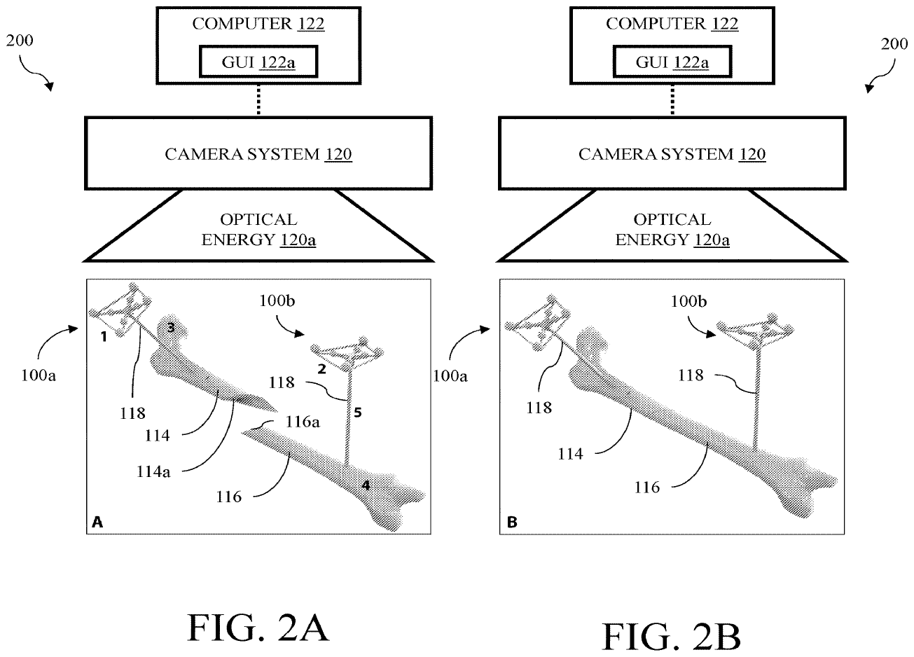

Referring now to FIGS. 2A-2B, a system 200 for orienting bone fragments is illustrated. System 200 is illustrated including a plurality of devices 100 (i.e., device 100a and device 100b). The description of device 100 in connection with FIGS. 1A-1B is applicable to devices 100a and 100b, unless indicated otherwise. Each of devices 100a and 100b are illustrated including an attachment device 118 (e.g., an orthopedic screw) used to attach each device to a respective bone fragment. First device 100a and second device 100b are configured (e.g., using an orthopedic screw) to be installed to a bone fragment (e.g., a proximal bone fragment, a distal bone fragments, etc.). First device 100a is illustrated attached to a proximal bone fragment 114. Second device 100b is illustrated attached to a distal bone 116.

In certain embodiments, device 100a can be the same as device 100b (e.g., having the same size, shape, orientation, etc.). In certain embodiments, device 100a can be different from device 100b (e.g., having a different frame size, having different sizes of one or more positional markers, having different sizes of one or more optical tracking markers, having a different attachment device, having a different shape, having a different orientation, and/or a combination thereof).

System 200 also includes a camera system 120. Camera system 120 is configured and adapted to detect one or more optical tracking markers (e.g., 102y1, 102y2, 102x1, and 102x2) of device 100a and device 100b. Camera system 120 is illustrated in electronic communication with computer 122 (e.g., a computer system, a desktop computer, a laptop computer, a personal computing device, etc.). Computer 122 is illustrated including a graphical user interface (GUI) 122a.

In FIG. 2A, camera system 120 is illustrated providing and/or receiving optical energy (e.g., visible light) prior to a repositioning operation. Camera system 120 is illustrated taking an image of device 100a and device 100b, including the optical tracking markers (e.g., 102y1, 102y2, 102x1, and 102x2) of each of device 100a and device 100b. Computer 122 can preprogramed to include details of each of device 100a and device 100b. For example, computer 122 can be preprogramed with the geometry of each of device 100a and device 100b (e.g., using a data file defining certain aspects of the geometry, using a 3D model, using a CAD model, etc.). Thus, computer 122 can determine a desired repositioning operation (e.g., a procedure by a medical professional to reposition proximal bone fragment 114 and distal bone fragment 116).

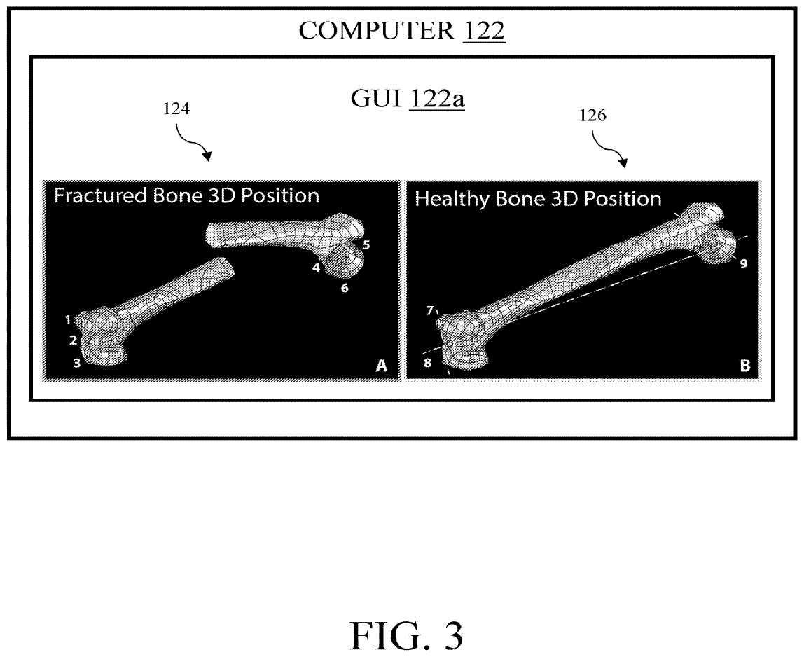

In certain embodiments, prior to determining a desired repositioning operation, a CT scan can be performed on the proximal bone fragment 114 and distal bone fragment 116. Such a CT scan (or another scanning operation, such as an X-ray) can be used to determine the geometry of the bone fragments (e.g., proximal bone fragment 114 and distal bone fragment 116). For example, a fracture profile 114a of proximal bone fragment 114 and/or a fracture profile 116a of proximal bone fragment 116 can be determined. Such a CT scan (or another scanning operation, such as an X-ray) can be used to determine the position of device 100a with respect to proximal bone fragment 114 (e.g., using one or more of the plurality of positional markers 104y1, 104y2, 104x1, and/or 104x2). Such a CT scan (or another scanning operation, such as an X-ray) can be used to determine the position of device 100b with respect to distal bone fragment 116 (e.g., using one or more of the plurality of positional markers 102y1, 102y2, 102x1, and/or 102x2). Such information (e.g., fracture profile 114a, fracture profile 116a, positional data, etc.) can be preloaded into computer 122 prior to an imaging operation of camera system 120 and/or a repositioning operation. Such information can be displayed graphically on GUI 122a (e.g., see FIG. 3). Medical personnel (e.g., a doctor, a surgeon, a nurse, etc.) can use such information prior to, during, or after a repositioning operation. In certain embodiments, camera system 120 can repeatedly take images and repeatedly update the GUI. For example, the position of proximal bone fragment 114 and distal bone fragment 116 can be repeatedly updated and displayed on GUI 122a.

Referring now to FIG. 2B, a camera system 120 is illustrated providing and/or receiving optical energy (e.g., visible light) after a repositioning operation is completed. Proximal bone fragment 114 and distal bone fragment 116 are illustrated in a desired position. Once medical personnel (e.g., a doctor, a surgeon, a nurse, etc.) determine the proximal bone fragment 114 and distal bone fragment 116 are a desired and final position, subsequent remedial or medical procedures can be conducted. For example, devices 100a and 100b can be removed (including removing attachment mechanisms 118) and a cast may be placed around the tissue surrounding the proximal bone fragment 114 and distal bone fragment 116. In another example, the position of the proximal bone fragment with respect to the distal bone fragment can be checked (e.g., via collecting one or more X-ray images). In certain embodiments, imaging technology (e.g., camera system 120) can be integrated into a surgical robotic system to drive the bone fragments into correct alignment.

Referring now to FIG. 3, computer 122 is illustrated displaying a 3D comparison between a fractured bone and a “healthy” bone. Image 124 displays a 3D scan of a distal and proximal fractured femur. Image 126 displays an intact bone of the contralateral femur. Such images can be generated by scanning a fractured bone (e.g., a fractured femur) and scanning an unfractured bone of the same patient (e.g., an unfractured femur from the same patient, where certain geometric aspects can be mirrored to achieve the “healthy bone” position). Such information can be used and continuously updated during a repositioning operation.

In certain applications, a surgeon can mark (e.g., using a touch screen GUI 122a) three landmarks on the distal part of a fractured bone (see reference markings “1”, “2”, and “3”) and three landmarks on the proximal part of the fractured bone (see reference markings “4”, “5”, and “6”). A surgeon can spot analogous (or the same) landmarks on the intact bone displayed in image 126; the surgeon can the mark such landmarks (e.g., using a stylus pen in connection with touch screen GUI 122a). An algorithm can be used to calculate the desired length and rotation of the femur based on the different axes: condylar axis (see reference marking “7”), mechanical axis (see reference marking “8”), and a neck axis (see reference marking “9”).

### Example 3

### Method for Orienting Bone Fragments

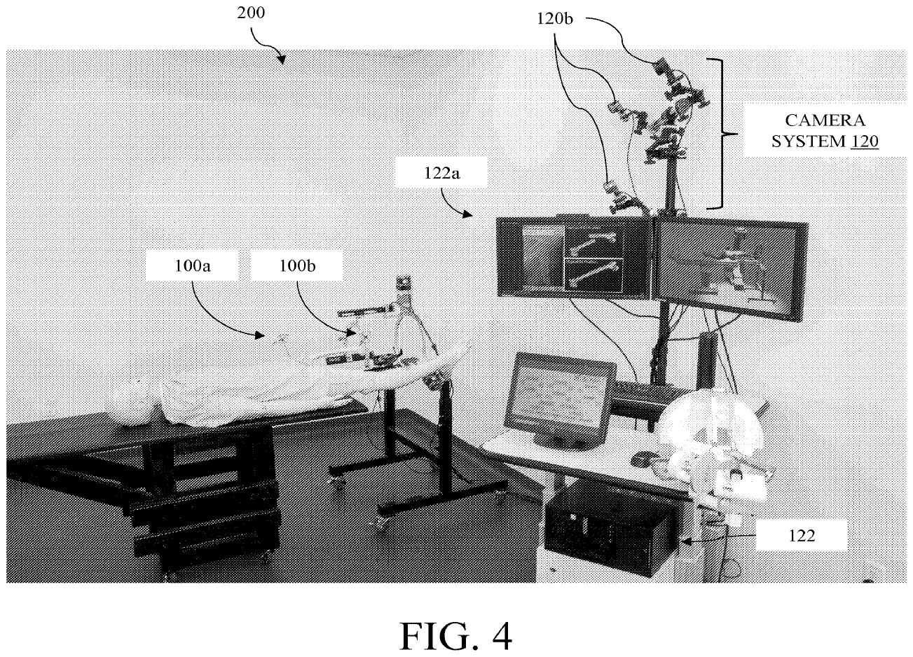

Referring now to FIG. 4, an embodiment of system 200 is illustrated in an experimental application. Camera 120 is illustrated including a plurality of cameras 120b.

In connection with system 200, an exemplary positioning (e.g., navigation) method is described in connection with a fractured femur. In a first step, a surgeon connects two uniquely designed rigid bodies (e.g., see FIG. 1A) to a distal and a proximal part of a fractured femur before surgery using a standard surgical screws (e.g., attachment mechanism 118 of FIGS. 2A-2B). Each rigid body can include four motion-tracking passive markers and four radiopaque sphere markers.

In a second step, a CT scan of the fractured femur is obtained with rigid bodies in the scene and also of the contralateral unfractured femur.

In a third step, a touch screen displays a GUI that shows the 3D scan of the healthy and fractured femur (e.g., see FIG. 3). The surgeon can manually input specific landmarks on the distal and proximal femur segments on the screen intraoperatively.

In a fourth step, using the input landmarks on the healthy bone, an algorithm calculates the lengths and rotations of the desired anatomical alignment.

In a fifth step, an optical tracking system (e.g., a NDI Polaris Vega XT) tracks the 3D position of the rigid bodies. To find the real-time spatial relative position of the fractured femur segments, optical tracking markers of the rigid bodies get registered to the radiopaque markers that are visible in the CT scan, considering the offsets. The surgeon can see the real-time 3D position of the bone parts on the GUI while manipulating the distal femur.

In a sixth step, the distal femur is attached to the moving ring of the robot via a standard surgical rod. Three methods are available to reduce the bone to its anatomical alignment based on the surgeon's desire: a) using the control panel to move the distal bone fragment in six degrees of freedom; b) manipulating the distal bone fragment (e.g., the distal femur) using the force-feedback controller while watching its real-time 3D position on the screen; and/or c) using an auto-alignment feature, which guides the robot to perform the reduction in an optimum path from the fractured position to the aligned position obtained from the healthy bone to achieve anatomical alignment.

In a seventh step, the surgeon can take X-ray images to check the bone position. After taking an X-ray image, the surgeon can change the approach and switch to another method (a, b, or c).

### Equivalents

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A system for orienting bone fragments comprising:
a first device including:
a first support element;
at least three first positional markers disposed on the first support element; and
at least three first optical tracking markers disposed on the first support element; and
a second device including:
a second support element;
at least three second positional markers disposed on the second support element: and
at least three second optical tracking markers disposed on the second support element:
wherein the first device is configured to be installed to a proximal bone fragment;
wherein the second device is configured to be installed to a distal bone fragment; and
wherein the first device and the second device are structurally different.

2. The system of claim 1, wherein the at least three first positional markers and the at least three second positional markers comprise tomography radiopaque materials.

3. (canceled)

4. (canceled)

5. The system of claim 2, wherein each of the positional markers is spherical.

6. The system of claim 2, further comprising a containment structure for each of the positional markers.

7. The system of claim 6, wherein the containment structure is configured to optically conceal each of the positional markers.

8. The system of claim 1, wherein the at least three first optical tracking markers and the at least three second optical tracking markers comprise infrared (IR)-detectable materials.

9. The system of claim 1, wherein:
the at least three first positional markers are fixed relative to the at least three first optical tracking markers on the first support element; and
the at least three second positional markers are fixed relative to the at least three second optical tracking markers on the second support element.

10. The system of claim 9, wherein:
the first optical tracking markers are radially distal to the first positional markers with respect to a substantially central point on the first support element; and
the second optical tracking markers are radially distal to the second positional markers with respect to a substantially central point on the second support element.

11. The system of claim 9, wherein:
at least one of the first positional markers is fixed in a different plane from at least one of the remaining first positional markers;
at least one of the first optical tracking markers is fixed in a different plane from at least one of the remaining first optical tracking markers;
at least one of the second positional markers is fixed in a different plane from at least one of the remaining second positional markers; and
at least one of the second optical tracking markers is fixed in a different plane from at least one of the remaining second optical tracking markers.

12. The system of claim 9, wherein:
at least one of the first positional markers is fixed asymmetrically in at least one dimension with respect to the remaining first positional markers;
at least one of the first optical tracking markers is fixed asymmetrically in at least one dimension with respect to the remaining first optical tracking markers;
at least one of the second positional markers is fixed asymmetrically in at least one dimension with respect to the remaining second positional markers;
at least one of the second optical tracking markers is fixed asymmetrically in at least one dimension with respect to the remaining second optical tracking markers; or
a combination thereof.

13. The system of claim 1, wherein the first support element and the second support element each comprise a fixed frame.

14. The system of claim 13, further comprising an orthopedic screw attached to the fixed frame.

15. The system of claim 14, further comprising a surgical robot coupled to the orthopedic screw attached to the second support element.

16. The system according to claim 1, further comprising a camera system configured and adapted to detect one or more optical tracking markers.

17. (canceled)

18. The system of claim 16, further comprising:
a computer system configured and adapted to communicate with the camera system, wherein the computer system is further configured and adapted to implement an optical tracking algorithm.

19. A method of monitoring long bone fragment positioning in real-time, the method comprising the steps of:
(a) providing the system according to claim 1;
(b) attaching the first device to the proximal long bone fragment;
(c) attaching the second device to the distal long bone fragment;
(d) obtaining imaging of the bone fragments with the attached first and second devices;
(e) determining a position of the first device with respect to the proximal long bone fragment based upon the imaging;
(f) determining a position of the second device with respect to the distal long bone fragment based upon the imaging;
(g) monitoring the first optical tracking markers of the first device and the second optical tracking markers of the second device using infrared (IR) cameras during at least a portion of a surgery; and
(g)-(h) determining a position of the distal long bone fragment relative to the proximal long bone fragment in real-time based upon the monitoring of the first and second optical tracking markers.

20. The method of claim 19, wherein the imaging in step (d) comprises a computed tomography (CT) scan or x-ray imaging.

21. The method of claim 19, wherein the surgery comprises manual or robotic surgery.

22. The method of claim 19, wherein the portion of the surgery in step (g) comprises a repositioning operation, the repositioning operation including moving the distal long bone fragment and relative to the proximal long bone fragment

23. The method of claim 19, further comprising:
(i) confirming a position of the proximal long bone fragment with respect to the distal long bone fragment.

24. The method of claim 23, wherein the confirming step includes collecting an X-ray image.
